(19) United States
(12) Patent Application Publication
(10) Pub. No.: US 2010/0221734 A1
(43) Pub. Date: Sep. 2, 2010

(54) SHOTGUN DNA MAPPING BY UNZIPPING

(76) Inventors: Steven J. Koch, Albuquerque, NM (US); Lawrence J. Herskowitz, Albuquerque, NM (US)

Correspondence Address: MUETING, RAASCH & GEBHARDT, P.A., P.O. BOX 581336, MINNEAPOLIS, MN, 55458-1336, US

(21) Appl. No.: 12/715,936

(22) Filed: Mar. 2, 2010

Related U.S. Application Data
(60) Provisional application No. 61/266,226, filed on Dec. 3, 2009.
(60) Provisional application No. 61/208,927, filed on Mar. 2, 2009.

(51) Int. Cl. C12Q 1/68

(52) U.S. Cl. 435/6

(57) ABSTRACT

The present invention provides a method of mapping a nucleic acid molecule such as, for example, DNA. Generally, the method includes providing a nucleic acid molecule comprising an unzipping force; comparing the unzipping force of the nucleic acid molecule to unzipping forces of a plurality of reference nucleic acid molecules, thereby generating a match score for each comparison; and identifying the reference nucleic acid that produces the best match score when compared to the nucleic acid molecule.

### CROSS-REFERENCE TO RELATED APPLICATION

This application claims priority to U.S. Provisional Patent Application Ser. No. 61/266,226, filed Dec. 3, 2009 and U.S. Provisional Patent Application Ser. No. 61/208,927, filed Mar. 2, 2009.

### GOVERNMENT FUNDING

This invention was made with Government support under grant number 0549500 awarded by the National Science Foundation. The U.S. Government has certain rights in this invention.

### BACKGROUND

Chromatin remodeling affects the ability of other proteins to access the DNA and can affect fundamental processes such as DNA repair and gene transcription by RNA polymerase. Understanding these dynamic remodeling processes requires the ability to characterize with high spatial and temporal resolution the changes to chromatin inside living cells. Techniques such as Chromatin Immunoprecipitation (ChIP), ChIP-chip, and other existing techniques have provided a wealth of important information, but have drawbacks in terms of sensitivity to small changes in protein occupancy, spatial resolution, and ensemble averaging.

### SUMMARY OF THE INVENTION

The present invention provides a method of mapping a nucleic acid molecule such as, for example, DNA. Generally, the method includes providing a nucleic acid molecule comprising an unzipping force; comparing the unzipping force of the nucleic acid molecule to unzipping forces of a plurality of reference nucleic acid molecules, thereby generating a match score for each comparison; and identifying the reference nucleic acid that produces the best match score when compared to the nucleic acid molecule.

In some embodiments, the method can include using reference nucleic acid molecules that map to known genomic locations—i.e., the genomic origin is known for each of the reference nucleic acid molecules in the organism from which the reference nucleic acid molecules are obtained. In such embodiments, the method can further include mapping the nucleic acid molecule to the genomic location of the best match reference nucleic acid molecule. In another aspect, the present invention provides a method for identifying a plurality of splice variants from an individual. Generally, the method includes receiving a biological sample from an individual, in which the biological sample includes a first splice variant comprising a first unzipping force, and a second splice variant comprising a second unzipping force; comparing the first unzipping force and the second unzipping force; and identifying the individual as possessing two different splice variants if the first unzipping force is not identical to the second unzipping force. In some embodiments, identifying certain splice variants can influence the selection of therapy that is likely to be most effective for the individual.

### BRIEF DESCRIPTION OF THE FIGURES

FIG. 1 illustrates shotgun DNA mapping by unzipping. A) The genome is digested into fragments, B) Each fragment is unzipped and the unzipping force is recorded, C) Experimental unzipping forces are compared to a library of simulated unzipping forces from a known genome.

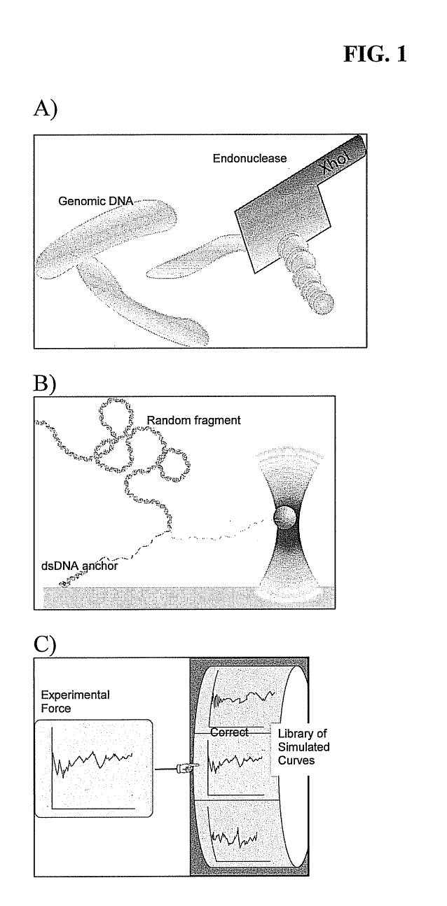

FIG. 2 shows an overview of the method for shotgun DNA and chromatin mapping.

FIG. 3 shows unzipping data comparing (A) correct and (B) incorrect simulation. The green window indicates the region from j=1200 to j=1700 where the match scores were computed. The increased separation of the two curves in the incorrect match is reflected in the higher match score of 0.8 versus 0.2 for the correct match.

FIG. 4 shows a compilation of match scores for a single experimental data set. The file number is arbitrary, arising from the order in which the library simulations were loaded. A perfect match would have a score of zero, and the correct match can be seen as having the lowest score, very distinguishable from the incorrect matches.

FIG. 5 shows a comparison of 32 match scores to all mismatch scores. The hatched histogram represents the match scores for the 32 experimental data sets, while the shaded histogram represents all incorrect match scores. Solid lines represent fits to the normal distribution. Overlap of the two distributions indicates probability of false positives.

FIG. 6 shows experimental optical tweezers unzipping of a single pBR322 molecule (dashed line, Koch 2002 data) compared with the simulated unzipping force (solid line, Herskowitz 2008). The parameters for the simulation are not optimized for this simulation data set. The match score is related to the amount of white space between the two curves (i.e., the less space, the better the match score).

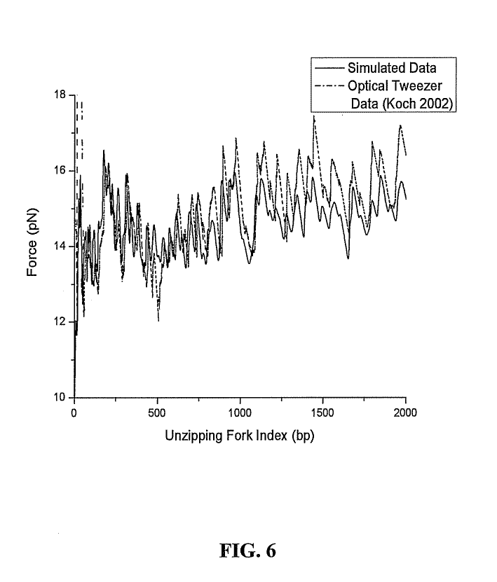

FIG. 7 illustrates the origins of energies used to create simulation libraries from known sequences. EDNA is the energy of base pairing and base stacking. EFJC is the energy from extending the single-stranded DNA using the freely-jointed chain model.

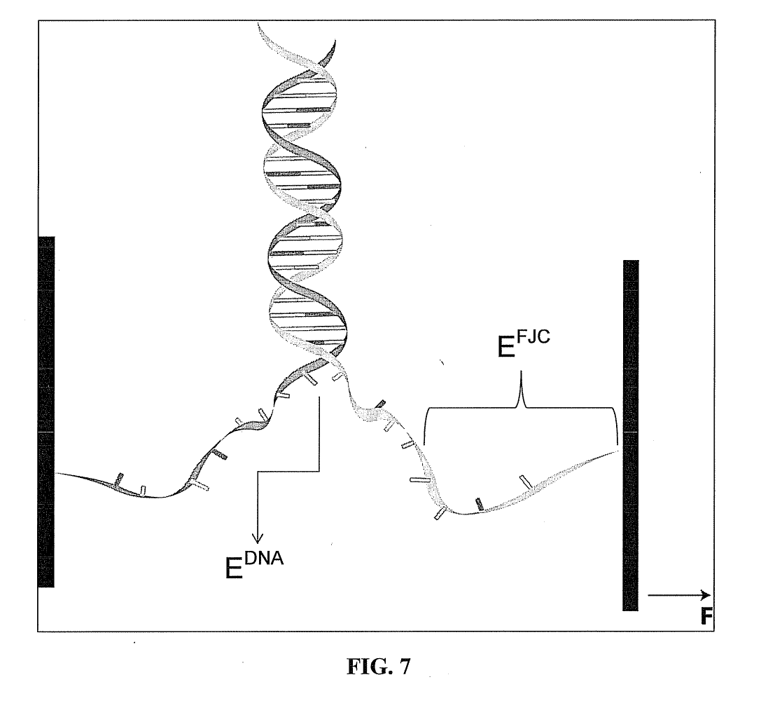

FIG. 8 shows a simulation curve obtained using the published sequence of pBR322.

FIG. 9 SDM analysis of expected unzipping signatures for c-Myb splice variants 8 and 8b.

FIG. 10 is a diagram of the complexity of the telomere structure.

### DETAILED DESCRIPTION OF ILLUSTRATIVE EMBODIMENTS

Shotgun DNA Mapping (SDM) enables one to identify the genomic location of a random DNA fragment based on its naked DNA unzipping forces compared with simulated unzipping forces of a published genome. By comparing the experimental unzipping forces to a library of simulated data from a known genome, match scores are obtained. The best match score indicates the most likely genome location for the unzipped fragment.

Generally, SDM can be performed by digesting genomic DNA with a site-specific endonuclease into random fragments. The random fragments are unzipped and force as a function of unzipping index is monitored and recorded. Force versus unzipping index data for all possible fragments of a known genome digested with the site-specific endonuclease can be computed, thereby forming a library of simulation data. The recorded experimental data is matched to the library and each experimental genome fragment can thereby be matched to a corresponding fragment in the library.

We demonstrate the utility of SDM by showing that 32 separate experimental unzipping curves for pBR322 were correctly matched to their simulated unzipping curves hidden in a background of the approximately 2700 sequences neighboring XhoI sites in the S. cerevisiae (yeast) genome.

### Definitions

SDM=Shotgun DNA Mapping;

SM=single-molecule;

ChIP=Chromatin Immunoprecipitation;

Pol II=RNA Polymerase II;

SCM=shotgun chromatin mapping

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

The ability to map polymerases and nucleosomes on chromatin is important for understanding the impact of chromatin remodeling on key cellular processes. Current methods (such as ChIP and ChIP-chip) have produced a wealth of information that demonstrates this importance, but key information is elusive in these ensemble methods. Additionally, other existing techniques have drawbacks in terms of sensitivity to small changes in protein occupancy, spatial resolution, and ensemble averaging. Certain information may be more readily, more accurately, and/or more precisely obtained via single-molecule (SM) analysis, such as, for example, seeing direct correlations between polymerases and nucleosomes on individual fibers or differentiating between some proposed models of chromatin remodeling.

To obtain this type of information, we have developed a single-molecule method for mapping polymerases and nucleosomes on chromatin based on optical tweezers unzipping of native chromatin molecules. SM DNA unzipping can map the positions of mononucleosomes assembled in vitro based on a repeatable nucleosome unzipping force profile. RNA Polymerase II (Pol II) complexes also may have a repeatable unzipping force profile, but may be distinguishable from nucleosomes and, therefore, may further provide information regarding, for example, the sense/antisense orientation of the Pol II. Thus, SM unzipping of native chromatin fragments—i.e., extracted from living cells—may provide high-resolution mapping of nucleosomes and Pol II molecules on individual chromatin fibers and, at least with respect to Pol II molecules, provide orientation information as well.

High-resolution SM mapping of individual fragments can provide information even if the specific location of the fragments in the genome is unknown. For example, the electron microscopy analysis of chromatin and RNA transcripts has demonstrated the utility of SM information even when the identity of the genes was unknown. However, it can be more powerful and thus desirable to obtain high-resolution SM information about specific genes or other sites of interest in the genome. For example, site-specific SM analysis may provide information regarding promoter-proximal Pol II pausing and antisense transcription.

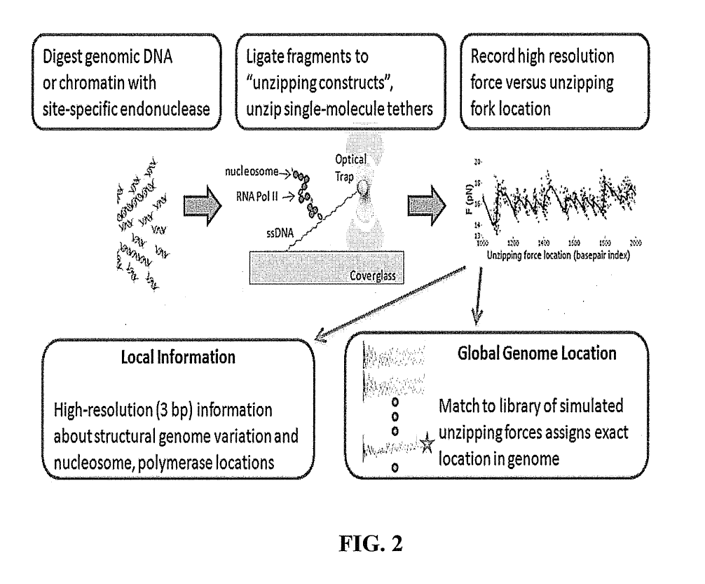

One approach for SM analysis involves unzipping random fragments of genomic DNA in a high-throughput fashion, and then determining from which specific site of the genome the unzipped fragment is located. We call this shotgun DNA mapping (SDM) and it is based on a method for indentifying the genomic location of naked DNA fragments (see FIG. 2). When applied to chromatin—i.e., genomic DNA associated with histone proteins—the method can be referred to as shotgun chromatin mapping (SCM).

SDM may be applied to DNA fragments from any source such as, for example, DNA from clone libraries, telomere restriction fragments, chromatin, and other sources. In SDM the genomic DNA is digested with a site-specific restriction endonuclease. The digestion may be performed using any suitable site-specific endonuclease that produces a known overhang upon digestion (i.e., sticky ends) or blunt end. In some embodiments, digestion with a restriction endonuclease that produces sticky ends can limit the number of simulations that must be performed because of the more limited number of sites recognized by such restriction endonucleases. In some embodiments, the digestion may be performed using XhoI, EcoRI, SapI, NotI, although it is possible to perform the digestion using any restriction endonuclease, hundreds of which are known and commercially available.

In some embodiments, the random fragments of genomic DNA may be derivatized by, for example, attaching the random fragments to one or more anchors such as, for example, a dsDNA anchor. In certain cases, the DNA fragments can be attached by ligating the genomic DNA fragments to the anchor. In other embodiments, the random fragments of genomic DNA may be derivatized by, for example, directly—i.e., in the absence of an anchor—attaching a chemical label such as, for example, digoxigenin or biotin to the genomic fragments.

The derivatized genomic fragments are unzipped and the force used to do so is measured. A derivatized genomic fragment may be unzipped by any suitable method such as, for example, by using any suitable single-molecule force apparatus. Suitable single-molecule force apparatuses include, for example, an optical tweezer, an atomic force microscope, a biomembrane force probe, or a magnetic tweezer may be used.

Unzipping forces for a known sequence of DNA can be accurately predicted by statistical mechanical models. Correlating experimental naked DNA unzipping forces with predicted unzipping forces of known DNA sequences allows one to identify the genomic location of random DNA fragments. We call this process shotgun DNA mapping (SDM). Generally, SDM includes comparing the unzipping force data for an unknown fragment to a library of simulated unzipping force data for known fragments. The unzipping force of a fragment of unknown DNA sequence reliably identifies a best match—i.e., the fragment from the library of known DNA sequences that includes the unknown fragment sequence. The identity of a DNA fragment can often be easily identified manually—e.g., by routine visual inspection—from among a handful of possibilities. Moreover, such comparisons may be accurate enough for automated identification of a fragment from the background of thousands of fragments that would be expected from site-specific digestion of genomic DNA.

The fragment possibilities can be limited, for example, by generating the random fragments by digesting genomic DNA with a site-specific restriction endonuclease.

SDM can have far reaching applications. SDM can be performed on any unknown fragment that can be attached to single-molecule force probes and is from a published genome. One characteristic of SDM is that it does not require genetic engineering or site-specific chromatin extraction.

SDM can be used to map the locations of nucleosomes on native chromatin, referred to herein as Shotgun Chromatin Mapping (SCM). Unzipping DNA through a nucleosome can locate the structure within 3 by resolution (Shundrovsky, A. et al. (2006). “Probing SWI/SNF Remodeling of the Nucleosome by Unzipping Single DNA Molecules.” Nature Structural and Molecular Biology, 13:549-554, doi:10.1038/nsmb1102). Generally, SCM involves digesting the chromatin with a site-specific endonuclease. The digestion fragments can be derivatized—i.e., attached to, for example, an anchor or a chemical label as described above—and unzipped. Unzipping removes the nucleosomes while recording the forces to do so. Relaxing the trap can allow the now naked ssDNA to reanneal. The reannealed naked DNA can then be unzipped. The naked DNA unzipping forces can be used in SDM, thereby allowing the locations of the original chromatin to be mapped.

More than 80% of all human genes are expressed as alternatively spliced mRNAs. (Matlin, A. et al. (2005). “Understanding alternative splicing: towards a cellular code.” Nature Reviews Molecular Cell Biology, 6:386-398, doi:10.1038/nrm1645). Moreover, approximately 60% of disease-causing mutations are alternative splice variants rather than changes in coding sequence. SDM can lead to rapid identification of different alternatively spliced mRNAs, each of which encodes a different protein isoform. Protein variants as a result of alternative splicing have been shown to be associated with many human diseases such as, for example, certain forms of cancer and several neurodegenerative disorders. In some cases, the existence of the splice variant and, therefore, identification of an individual as possessing the alternative splice variant, may be associated with the individual exhibiting one or more symptoms or clinical signs of the condition. As used herein, a splice variant “associated” with a human condition is a splice variant found to be present in a “affected” sample—e.g., an individual or a particular organ, tissue, and/or cell affected by the condition such as, for example, a tumor, tumor cell, or degenerative neuron—in greater amounts and/or with greater frequency than it is found to be in a normal sample. A splice variant may be “associated” with a condition regardless of whether the splice variation is a causative agent in the development of the condition or is a secondary indication of the condition. In other cases, the existence of the splice variant may indicate that the individual is predisposed to a condition even though the individual may not exhibit a symptom or clinical sign of the condition. As used herein, an individual “predisposed” to a condition is an individual that possesses an alternative splice variant known to be associated with an increased risk of developing the condition. An individual may be predisposed to a condition regardless of whether the individual exhibits one or more symptoms or clinical signs of the condition at the time the predisposition is identified.

One example of a specific splicing variant associated with a cancer is human DNA methyltransferase (DNMT) genes. Three DNMT genes encode enzymes that add methyl groups to DNA, a modification that often has regulatory effects. Several abnormally spliced DNMT3B mRNAs are found in tumors and cancer cell lines. Cells expressing abnormal DNMT mRNAs exhibited changes in DNA methylation patterns and/or grew twice as fast as control cells, indicating a direct contribution to tumor development by this product.

Another example of a specific splicing variant associated with a cancer is the Ron macrophage-stimulating protein receptor (MSTJR) proto-oncogene. One property of cancerous cells is their ability to move and invade normal tissue. Production of an abnormally spliced transcript of Ron is associated with increased levels of the splicing factor SF2/ASF in breast cancer cells. The abnormal isoform of the Ron protein encoded by this mRNA leads to increased cell motility.

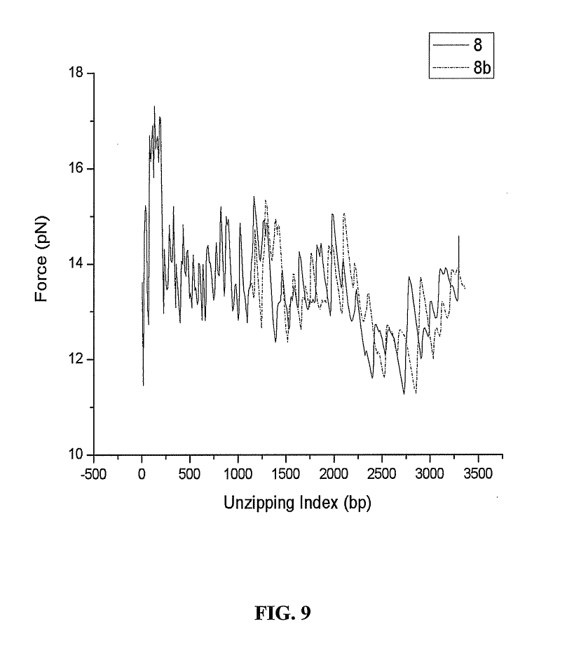

In addition, identifying alternative splice variants in an individual can provide important diagnostic information. An example of this technique is shown in FIG. 9. This figure shows the predicted unzipping signature of cDNA from two different splice variants of c-Myb protein. Certain variants of the c-Myb protein are involved in the development of leukemia. Thus, SDM mapping can be used to identify individuals having a genetic predisposition for certain conditions or to characterize varieties of splice variants in tumor cells. The genetic predisposition and/or tumor cell splice variant infoimation can be used to predict the efficacy of various therapies such as, for example, chemotherapeutics that may be more effective against certain forms of the condition.

Using SDM to identify alternative splice variants is not limited to the particular splice variants or particular conditions identified immediately above. Rather, SDM can be a general technique suitable for identifying any splice variant found to be associated with any condition.

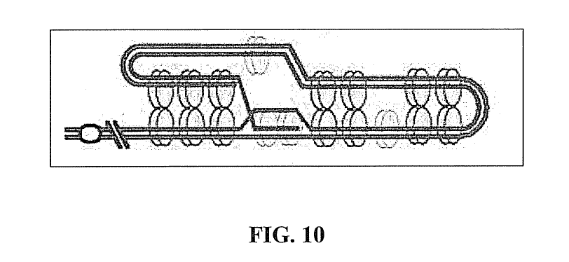

Also, SDM can provide a new technique for studying telomere structure. The repetitive nature of telomeres makes it difficult to study under many methods. SDM unzipping technique works extremely well with repetitive sequences and known endonuclease sites before the telomeric region starts might allow SDM to work well to study its structure. FIG. 10 is a diagram of the complexity of the telomere structure specifically showcasing the t-loop and a possible mode for t loop formation. T loops might be used to protect the telomere termini from cellular activity.

SDM can also be used to perform structural genome mapping. Structural genome changes can be a feature of genetic variation. (Kidd, J. M. et al. (2008) “Mapping and sequencing of structural variation from eight human genomes.” Nature, 453(7191), 56-64. Nature Publishing Group. doi: 10.1038/nature06862.) Genome inversions, deletions, and insertions can perturb the unzipping signal in a manner similar to the effect of the deletion/insertion depicted in FIG. 9. SDM provides a single-molecule method for characterizing structural genome changes and revealing heterogeneity such as, for example, haplotype or an array of mutations in cancer cells.

We show herein that SDM is possible. Specifically, we demonstrate that the modeling of the pBR322 unzipping forces is sufficiently accurate so that experimental data are successfully matched to the pBR322 sequence hidden in a background of the approximately 2700 XhoI fragments from the S. cerevisiae genome. SDM may provide a platform shotgun chromatin mapping. Furthermore, we envision other high impact applications, for example single-molecule structural genome mapping (Kidd et al. (2008)) and new assays for screening protein binding sites by shotgun DNA mapping in the presence of purified proteins.

### Methods

All computations below were carried out using a Dell duoCore running Windows XP, using code written in LabVIEW 7.1.

### Experimental Single-Molecule Unzipping Data

We obtained force (F) versus unzipping index (j) for 32 data sets of unzipping pBR322 from the published data of Koch et al. (Koch, S. J et al., “Probing protein-DNA interactions by unzipping a single DNA double helix.” Biophys J 83: 1098 (2002)). Data were obtained and analyzed with optical tweezers and unzipping constructs as described. (Data acquisition software available on openwetware. Data analysis performed as described herein below.) The format of these data sets is tab delimited text files, with the “Force (pN)” and “index (j)” columns used by us. The 32 raw data sets are available on http://kochlab.org. We used particular data sets which seem to have significant viscous drag due to high stretching rate.

Data were smoothed according to a sliding boxcar smoothing algorithm we implemented in LabVIEW. We used a 30 point window with equal weighting to each point in the window, and a window step size of j=1. Smoothed data sets were stored in text files of the same format as the simulated data (below) and are available on http://kochlab.org or upon request.

### Extraction of Yeast Genome XhoI Sites

We obtained the yeast genome (S. cerevisiae) from yeastgenome.org. We downloaded a text file for each chromosome of the yeast genome. XhoI recognition sites (CTGCAG) were identified. Each XhoI recognition site defined two fragments. One fragment included 2000 by upstream of the XhoI recognition site, the other includes 2000 by downstream of the XhoI recognition site. The upstream fragments were reversed so that they begin with the XhoI recognition site. Also, pBR322 fragments generated from digestion of the plasmid with Earl were added to the fragment library.

### Creation of Simulation Library for Yeast XhoI Sites

It has been shown that the unzipping forces for a known sequence of DNA can be accurately predicted by statistical mechanical models (Bockelmann, U., et al., “Molecular stick-slip motion revealed by opening DNA with piconewton forces.” PHYSICAL REVIEW LETTERS 79: 4489 (1997)).

The expectation values for force and unzipping index (as seen below) form the simulated curves that will be compared to the experimental data.

$$\begin{matrix}〈 F 〉 = {\sum }_{ }^{ } {F}_{j} {P}_{j} = \frac{{\sum }_{ }^{ } {F}_{j} {e}^{- \frac{{H}_{j}}{{k}_{b} T}}}{{\sum }_{ }^{ } {e}^{- \frac{{H}_{j}}{{k}_{b} T}}} & Formula 1 \\ 〈 j 〉 = {\sum }_{ }^{ } j {P}_{j} = \frac{{\sum }_{ }^{ } j {e}^{- \frac{{H}_{j}}{{k}_{b} T}}}{{\sum }_{ }^{ } {e}^{- \frac{{H}_{j}}{{k}_{b} T}}} & Formula 2\end{matrix}$$

where P stands for probability, F is the force, Hj is the Hamiltonian at a specific base pair j, and k_{b}T is the thermal energy.

The Hamiltonian needed to generate the expectation values relies on the energy from base pairing and from the ssDNA.

Hⱼ=Eⱼ^{DNA}+Eⱼ^{FJC} Formula 3:

where EDNA is the energy required to break the base pair bonds. EFJC is the energy from the freely jointed chain.

$$\begin{matrix}{E}_{j}^{DNA} = {\sum }_{i}^{j} {E}_{i} & Formula 4 \\ {E}_{j}^{FJC} = xF - \int x ( {F}^{′} ) d {F}^{′} & Formula 5 \\ {E}_{i} = \{ \begin{matrix}1.4 {k}_{B} T , & for A \text{-} T \\ 2.9 {k}_{B} T , & for G \text{-} C\end{matrix} & Formula 6 \\ x ( F ) = {L}_{0 } [ 1 - \frac{1}{2} {( \frac{{k}_{B} T}{{FL}_{P}} )}^{1 / 2} + \frac{F}{K} ] & Formula 7\end{matrix}$$

1.4 k_{B}T and 2.9 k_{B}T (Bockelmann 1997) where used for A-T and G-C base pairing respectively. LO is the contour length which was 0.54 nm per nt. LP is the persistence length, 0.8 nm, and K stands for the stretch modulus, 580 pN (Koch 2002).

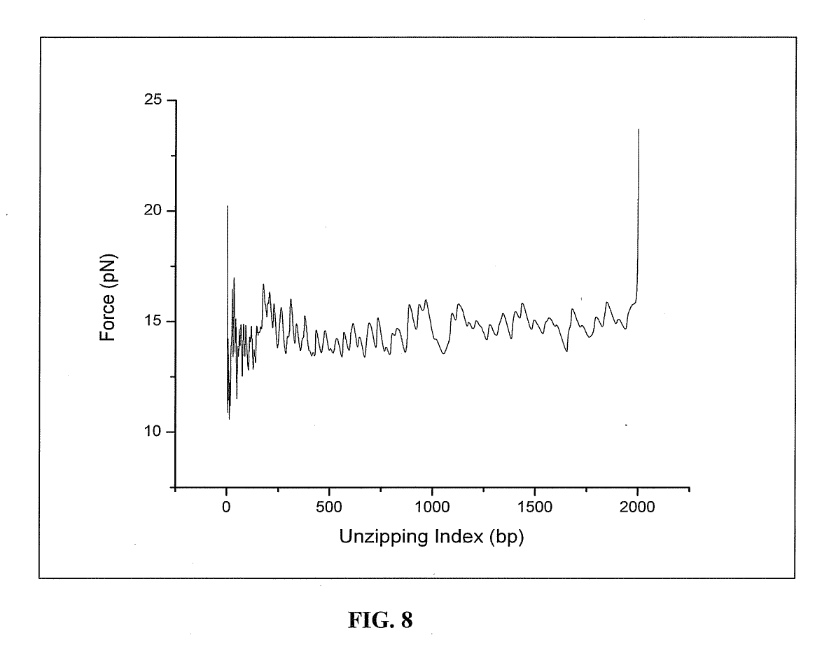

An example of a simulation curve (FIG. 8) was obtained based on the math above and the pBR322 sequence as published.

We created a library of simulation for XhoI digestion of the S. cerevisiae genome. The sequence of this genome was obtained from yeastgenome.org. XhoI recognition sites, CTCGAG, were searched for inside the yeast genome. For each recognition site two fragments were formed, 2000 base pairs before the site and 2000 base pairs after. This process produced 2,784 fragments. For each fragment the sequence was loaded and the expectation values were calculated in steps of 1 nm from 1 nm to 2200 nm and sum over j from 1 to 2000. Additionally the pBR322 sequence used in Koch 2002 was manually added to the sequence library with a code name to blind it from the data analyzers.

Expectation values for F, j, and the variance of each for a given DNA sequence and end to end length, l, were calculated as simple sums over all possible j values (from 1 to the length of the sequence). Simulated F versus j curves were then generated by repeating the calculation over varying values for l. An automated process loaded each sequence and produced F versus j curves for all yeast XhoI fragments in the library. For this work, the expectation values were calculated in steps of 1 nm from 1 nm to 2200 nm and sums over j from 1 to 2000. Simulation results were stored in text files, one file for each XhoI fragment and will be available from http://kochlab.org.

### Matching Algorithms

We compared the force versus j curve for an unknown fragment and the computed force versus j curves in the library of fragments with known sequences. We call the measure of this comparison the match score (m), and it is derived from the standard deviation of the two curves in a given interval. To compute m we used Formula 8.

$$m = \frac{1}{\sqrt{N}} \frac{\sqrt{{\sum }_{i}^{N} {( 〈 {F}_{i}^{exp} 〉 - 〈 {F}_{i}^{sim} 〉 )}^{2}}}{{F}_{G} - {F}_{A}} \qquad Formula 8$$

where N is the number of points in the window, F^{exp} and F^{sim} are the experimental and simulated unzipping forces, respectively. FG and FA are the maximum and minimum forces attained from DNA sequences filled with only G and A base pairs, respectively. FG was found to be 17.561 pN, and FA was 10.227 pN. The maximum possible score for mismatch is thus 1. While a perfect match is a score of 0.

### Robustness Analysis

We created a histogram of all incorrect match scores (noise) and fit the histogram to a Gaussian using OriginPro (OriginLab, Northampton, Mass.). A second histogram for all correct match scores was created, and also fit to a Gaussian using OrignPro. An estimate of the robustness was produced by comparing the difference of the means of signal to noise relative to the standard deviation of the noise.

### Results

### Experimental Single-Molecule Unzipping Data

We smoothed 32 data sets for unzipping of an Earl fragment of pBR322. Examination of force versus unzipping index shows a noticeable increase in the unzipping force for j>1000. This is due to a significant increase in the unzipping rate above j=1000, because the original purpose of these data sets (Koch 2002) was to probe protein occupancy, where an increased unzipping rate is desirable and a systematic shift in unzipping force is not an issue.

### Extraction of Yeast Genome XhoI Sites

We found approximately 1350 XhoI sites in the yeast genome, which produced a library of approximately 2700 upstream and downstream unzipping fragments. The entire search and extraction took only a few minutes on our platform. Fewer than 10 XhoI sites were within 2000 by from the end of the chromosome, producing fragments less than the desired 2000 bp. These fragments produced nonsense match scores, which were then discarded for these test purposes. Also, by chance, some XhoI sites were separated by less than 2000 basepairs, and thus some fragments included XhoI recognition sequences. In an actual shotgun DNA mapping experiment, these XhoI sites could produce shortened fragments, depending on the level of completion of digestion. Close neighboring sites and sites near chromosome ends can be dealt with in certain embodiments of SDM, but for this purpose it was not necessary to use those methods. The resulting library (will be available on http://kochlab.org) included the hidden pBR322 fragments.

### Creation of Simulation Library for Yeast XhoI Sites

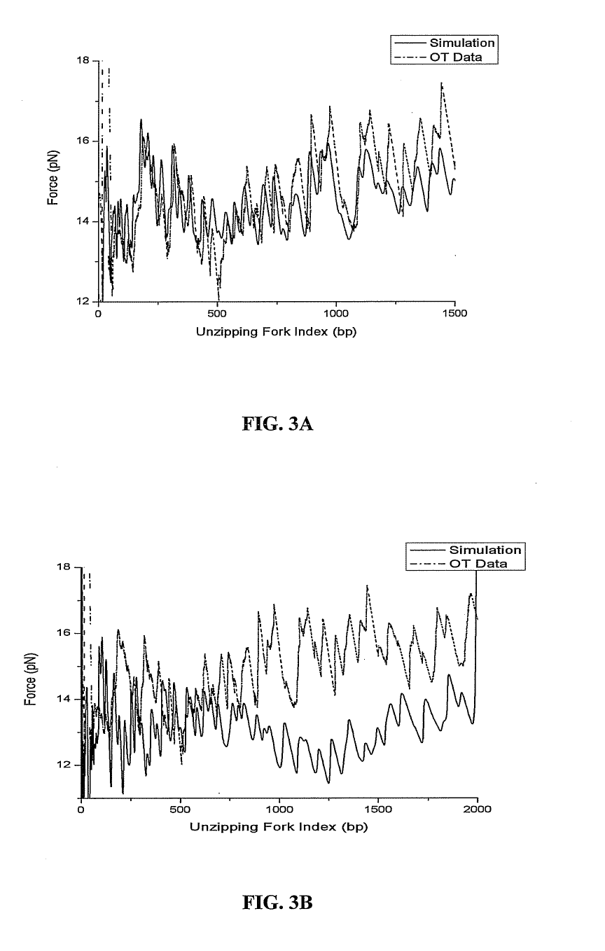

The force (f) versus unzipping index (j) was simulated for every fragment in the sequence library from l=1 nm to 2200 nm. Simulation of approximately 2700 files took approximately a few days on our computational platform. Examples of these simulated curves can be seen in FIG. 3A and FIG. 3B. Simulations were stored in a library of tab delimited text files.

### Matching Experimental Data and Library Fragments

One feature of the shotgun DNA mapping process is a mechanism for producing a quantitative number comparing an experimental data set and an entry in the simulation library. We first attempted a cross-correlation algorithm (as in Shundrovsky et al., “Probing SWI/SNF remodeling of the nucleosome by unzipping single DNA molecules.” Nature Structural & Molecular Biology 13:549 (2006)), which was unsatisfactory due to the insensitivity of cross-correlation to vertical shifts. That is, the cross-correlation score does not change if the simulation forces are scaled by a factor of 10, for example. Because the unzipping forces reflect the energy of the DNA basepairing, which is directly related to the DNA sequence, absolute unzipping force is an important factor in identifying an unknown fragment. Thus, we developed a method based on the standard deviation between the two curves, as described in the methods.

Window size

Referring to FIG. 3A and FIG. 3B, the shaded boxes highlight the window over which the match scores were computed (j=1200 to 1700). There were a number of reasons for choosing this window size and location. For some shotgun DNA mapping applications, it will be desirable to have the matching window as close to the initial unzipping sequence as possible. However, our current implementation of the DNA unzipping simulation accounts for neither the optical tweezers compliance nor the compliance of the 1.1 kilobases of dsDNA that was used to anchor the segment to the coverglass. This added compliance can influence the data obtained for the initial unzipping region, where the length of single-stranded DNA is relatively low and thus much stiffer. Furthermore, the data sets we are using have a discontinuous unzipping rate, switching at j≈1000 from a slow unzipping rate with large data averaging to a fast unzipping rate with no data averaging. Thus, we have selected the window to lie on either side of this transition. Neither side is ideal (too much averaging for j<1000 and viscous drag for j>1000), which may demonstrate the robustness of our method. We chose j>1000 because of the decreased amount of averaging associated with the j<1000 raw data.

The ability to use a smaller window size is also desirable for shotgun mapping applications. We investigated the results of smaller window sizes and found that smaller windows (for example 100 base pairs wide) produced results that were more dependent on the overall location of the window (results from poor to just as good as we show here, data not shown). In contrast, the 500 base pair window was relatively insensitive to location. We chose to use the 500 base pair window so that window location would not significantly affect our results.

This test used experimental unzipping data that was not produced for the purpose of SDM. Embodiments of SDM can produce data that is optimized for SDM and thus smaller windows and other window locations will work. Data may be optimized for SDM by, for example, eliminating viscous drag, reducing drift, and reducing noise. Furthermore, SDM embodiments can include a known stretch of DNA before the unknown fragment. This known stretch can be used to subtract off drift and other systematic errors for every data set. This will greatly increase the robustness of the method.

### Shotgun Mapping Results

FIG. 3A and FIG. 2B [double check FIGS!!!] show a comparison of the F versus j curves for the correct match as well as an incorrect match, respectively. By eye, it can easily be seen that there is a larger deviation between the two curves in the FIG. 2B. This is reflected by the increased white space between the curves, which is, effectively, quantified by the match score (m). Thus, a score of zero reflects a perfect match. For this particular data set, the match score was 0.2, and the mismatch shown produced a score of 0.8.

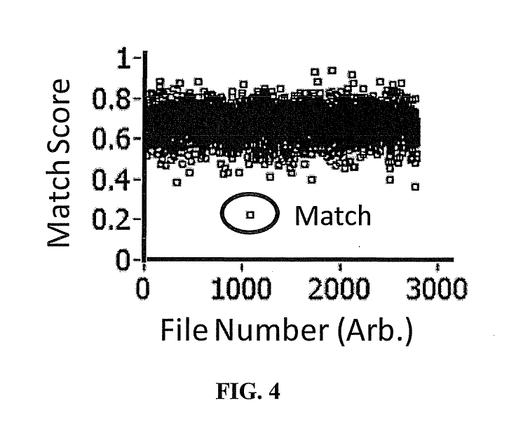

The match scores for this experimental curve against the entire library are shown in FIG. 4. In order to prevent biasing our initial assessments of our method, we produced this figure blindly, with the identity of the correct match unknown to the operator of the shotgun mapping application. We found that one match score fell far below the mean of all match scores (5σ away), and was significantly lower than even the next best match score. At this point, we unblinded the file number of the correct match, the pBR322 simulation and confirmed that our method successfully identified the experimental fragment, based on the criteria of best match score.

### Robustness Analysis

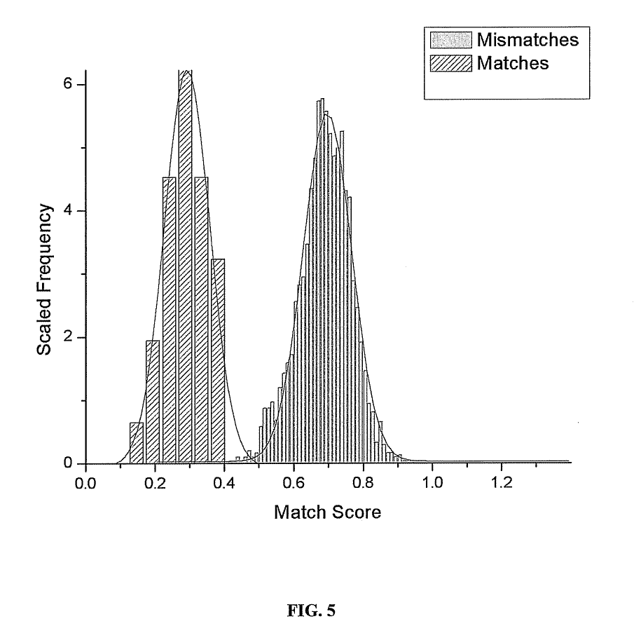

FIG. 4 shows successful shotgun DNA mapping for one of the experimental data sets. We repeated this for all 32 data sets and the correct match was the best score in every case. We did not find any instance of incorrect assignment for the window size and location we chose. To better visualize the robustness, we created histograms of all the scores for all the matches (N=32) and all the mismatches (N≈2700*32) and fit these histograms to Gaussian functions. These data are shown in FIG. 5, with the correct matches in blue and the mismatches in red. The integrated area of overlap between the two Guassian fits is a small number, another indicator of the expected rate of false positives. The only overlap is in the tails of the Gaussians, a region where it is likely that the true experiments would significantly differ from a normal distribution, so this only provides an estimate of the true error rate.

The robustness shown in FIG. 4 is somewhat surprising, given the effect of viscous drag on the experimental unzipping forces. We found that the match scores relative to the mismatches was not much different for these data sets, compared to one data set we obtained without the viscous drag effect (data not shown). A possible explanation for this is that the pBR322 sequence has high GC content in the comparison region, and thus a vertical shift of the data merely tends to shift both the correct matches and the mismatches to higher values, without increasing the overlap of the two histograms shown in FIG. 5.

These results indicate the feasibility of performing SDM of yeast genomic DNA using, for example, restriction fragments generated by XhoI digestion. Based on our results, SDM is effective employing libraries such as those of the size generated by digesting the genome of S. cerevisiae with a restriction endonuclease having a 6 by recognition site. Larger libraries may be generated by using either a larger genome and/or a restriction endonuclease having a shorter recognition sequence. Conversely, smaller libraries may be generated using either a smaller genome or a restriction endonuclease having a larger recognition sequence. Although the above description refers to an exemplary method using S. cerevisiae, those skilled in the art will recognize that embodiments are not limited to S. cerevisiae. Other applications include, but are not limited to, structural genome mapping in humans and other organisms; mapping of single cells (e.g., mapping genome rearrangement in human cancer cells); screening human and other genomes for protein binding sites; and screening for small molecules that can disrupt or enhance protein binding.

Other embodiments include, but are not limited to, the formation of single-molecule tethers using XhoI-digested yeast DNA and shotgun chromatin mapping which will complement the single-site plasmid chromatin experiments.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### REFERENCES

1. Valouev, A., Schwartz, D., Zhou, S., Waterman, M., “An algorithm for assembly of ordered restriction maps from single DNA molecules.” Proceedings of the National Academy of Sciences 103, 15770 (2006).
2. Lin, J., Qi, R., Aston, C., Jing, J., Anantharaman, T. S., Mishra, B., White, O., Daly, M. J., Minton, K. W., Venter, J. C., Schwartz, D. C., “Whole-genome shotgun optical mapping of Deinococcus radiodurans.” Science (New York, N.Y.) 285, 1558 (1999).
3. Samad, A., Huff, E. F., Cai, W., Schwartz, D. C., “Optical mapping: a novel, single-molecule approach to genomic analysis.” Genome research 5, 1 (1995).
4. Cai, W., Aburatani, H., Stanton, V. P., Housman, D. E., Wang, Y. K., Schwartz, D. C., “Ordered restriction endonuclease maps of yeast artificial chromosomes created by optical mapping on surfaces.” PROCEEDINGS OF THE NATIONAL ACADEMY OF SCIENCES OF THE UNITED STATES OF AMERICA 92, 5164 (1995).
5. Schwartz, D. C., Li, X., Hernandez, L. I., Ramnarain, S. P., Huff, E. J., Wang, Y. K., “Ordered restriction maps of Saccharomyces cerevisiae chromosomes constructed by optical mapping.” Science (New York, N.Y.) 262, 110 (1993).
6. Kidd, J., Cooper, G., Donahue, W. et al., “Mapping and sequencing of structural variation from eight human genomes.” Nature 453, 56.
7. Boeger, H., Griesenbeck, J., Kornberg, R. D., “Nucleosome retention and the stochastic nature of promoter chromatin remodeling for transcription.” Cell 133, 716 (May 16, 2008).
8. Shundrovsky, A., Smith, C. L., Lis, J. T., Peterson, C. L., Wang, M. D., “Probing SWI/SNF remodeling of the nucleosome by unzipping single DNA molecules.” Nature Structural & Molecular Biology 13, 549 (2006).
9. Osheim, Y. N., Sikes, M. L., Beyer, A. L., “EM visualization of Pol II genes in Drosophila: most genes terminate without prior 3′ end cleavage of nascent transcripts.” Chromosoma 111, 1 (2002).
10. He, Y., Vogelstein, B., Velculescu, V., Papadopoulos, N., Kinzler, K., “The Antisense Transcriptomes of Human Cells.” Science, 1163853 (2008).
11. Core, L., Waterfall, J., Lis, J., “Nascent RNA Sequencing Reveals Widespread Pausing and Divergent Initiation at Human Promoters.” Science, 1162228 (2008).
12. Buratowski, S., “TRANSCRIPTION: Gene Expression—Where to Start?” Science 322, 1804 (2008).
13. Margaritis, T., Holstege, F. C., “Poised RNA polymerase II gives pause for thought.” Cell 133, 581 (2008).
14. Muse, G., Gilchrist, D., Nechaev, S., Shah, R., Parker, J., Grissom, S., Zeitlinger, J., Adelman, K., “RNA polymerase is poised for activation across the genome.” Nature Genetics 39, 1507 (2007).
15. Zeitlinger, J., Stark, A., Kellis, M., Hong, J. W., Nechaev, S., Adelman, K., Levine, M., Young, R. A., “RNA polymerase stalling at developmental control genes in the Drosophila melanogaster embryo.” Nat Genet 39, 1512 (December 2007).
16. Core, L., Lis, J., “Transcription Regulation Through Promoter-Proximal Pausing of RNA Polymerase II.” Science 319, 1791 (2008).
17. Bockelmann, U., Thomen, P., Essevaz-Roulet, B., Viasnoff, V., Heslot, F., “Unzipping DNA with optical tweezers: high sequence sensitivity and force flips.” BIOPHYSICAL JOURNAL 82, 1537 (2002).
18. Bockelmann, U., EssevazRoulet, B., Heslot, F., “Molecular stick-slip motion revealed by opening DNA with piconewton forces.” PHYSICAL REVIEW LETTERS 79, 4489 (1997).
19. Koch, S. J., Shundrovsky, A., Jantzen, B. C., Wang, M. D., “Probing protein-DNA interactions by unzipping a single DNA double helix.” Biophys J 83, 1098 (August 2002).
20. SantaLucia, J., Jr., “A unified view of polymer, dumbbell, and oligonucleotide DNA nearest-neighbor thermodynamics.” PNAS 95, 1460 (Feb. 17, 1998, 1998).
21. Cai, W., Jing, J., Irvin, B., Ohler, L., Rose, E., Shizuya, H., Kim, U. J., Simon, M., Anantharaman, T., Mishra, B., Schwartz, D. C., “High-resolution restriction maps of bacterial artificial chromosomes constructed by optical mapping.” PROCEEDINGS OF THE NATIONAL ACADEMY OF SCIENCES OF THE UNITED STATES OF AMERICA 95, 3390 (1998).

What is claimed is:

1. A method comprising:
receiving a nucleic acid molecule comprising an unzipping force;
comparing the unzipping force of the nucleic acid molecule to unzipping forces of a plurality of reference nucleic acid molecules, thereby generating a match score for each comparison; and
identifying the reference nucleic acid that produces the best match score when compared to the nucleic acid molecule.

2. The method of claim 1 wherein each of the plurality of reference nucleic acid molecules maps to a known genomic location.

3. The method of claim 2 further comprising mapping the nucleic acid molecule to the genomic location of the best match reference nucleic acid molecule.

4. The method of claim 1 wherein providing a nucleic acid molecule comprises digesting genomic DNA with a restriction endonuclease, thereby producing at least one nucleic acid molecule comprising a restriction fragment of the genomic DNA.

5. The method of claim 4 wherein digesting the genomic DNA with the restriction endonuclease produces at least one nucleic acid molecule that comprises a 5′ overhang.

6. The method of claim 4 wherein the restriction endonuclease comprises XhoI, EcoRI, SapI, or NotI.

7. The method of claim 1 further comprising attaching at least one nucleic acid molecule to an anchor.

8. The method of claim 7 wherein attaching at least one nucleic acid molecule to an anchor comprises ligating the nucleic acid molecule to dsDNA.

9. The method of claim 1 further comprising attaching at least one nucleic acid molecule to a chemical label.

10. The method of claim 9 wherein the chemical label comprises digoxigenin or biotin.

11. The method of claim 1 further comprising measuring the unzipping force of at least one nucleic acid molecule.

12. The method of claim 11 wherein the unzipping is measured using a single-molecule force apparatus.

13. The method of claim 12 wherein the single-molecule force apparatus comprises an optical tweezer, an atomic force microscope, a biomembrane force probe, or a magnetic tweezer.

14. A method for identifying a plurality of splice variants from an individual, the method comprising:
receiving a biological sample from an individual comprising:
a first splice variant comprising a first unzipping force, and
a second splice variant comprising a second unzipping force;
comparing the first unzipping force and the second unzipping force; and
identifying the individual as possessing two different splice variants if the first unzipping force is not identical to the second unzipping force.

15. The method of claim 14 wherein at least one of the splice variants is associated with a condition or a genetic predisposition for the condition.

16. The method of claim 15 wherein the condition comprises a cancer associated with an alternative splice variant.

17. The method of claim 15 further comprising selecting a therapy effective for the condition associated with at least one identified alternative splice variant.

18. The method of claim 17 further comprising administering an effective amount of the therapy to the individual.

19. The method of claim 15 wherein the at least one splice variant comprises a splice variant of human DNA methyltransferase, macrophage-stimulating protein receptor, or c-Myb.
